SEQUENCE LISTING (8 sequences)

SEQ ID NO: 1 - protein, 454 aa, synthetic construct
  REGION 1..454: note = Antibody heavy chain
EVKLVESGGGLVQPGGSLRLSCAASGFTFNTYAMNWVRQAPGKGLEWVARIRSKYNNYATYYADSVKDRFTISRDDSKSSLYLQMNNLKTEDTAMYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFLLYSKLTVDKSRWQQGNVFSCSVMHEALHNHYTQKSLSLSPG

SEQ ID NO: 2 - protein, 215 aa, synthetic construct
  REGION 1..215: note = Antibody light chain
QAVVTQEPSFSVSPGGTVTLTCRSSTGAVTTSNYANWVQQTPGQAFRGLIGGTNKRAPGVPARFSGSLIGDKAALTITGAQADDESIYFCALWYSNLWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 3 - protein, 451 aa, synthetic construct
  REGION 1..451: note = Antibody heavy chain
EVQLVESGGGLVQPDRSLRLSCAASGFTFHDYAMHWVRQAPGKGLEWVSTISWNSGTIGYADSVKGRFTISRDNAKNSLYLQMNSLRAEDTALYYCAKDIQYGNYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPG

SEQ ID NO: 4 - protein, 214 aa, synthetic construct
  REGION 1..214: note = Antibody light chain
EIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 125 aa, synthetic construct
  REGION 1..125: note = Heavy chain variable region sequence l
EVKLVESGGGLVQPGGSLRLSCAASGFTFNTYAMNWVRQAPGKGLEWVARIRSKYNNYATYYADSVKDRFTISRDDSKSSLYLQMNNLKTEDTAMYYCVRHGNFGNSYVSWFAYWGQGTLVTVSS

SEQ ID NO: 6 - protein, 109 aa, synthetic construct
  REGION 1..109: note = Light chain variable region sequence
QAVVTQEPSFSVSPGGTVTLTCRSSTGAVTTSNYANWVQQTPGQAFRGLIGGTNKRAPGVPARFSGSLIGDKAALTITGAQADDESIYFCALWYSNLWVFGGGTKLTVL

SEQ ID NO: 7 - protein, 122 aa, synthetic construct
  REGION 1..122: note = Heavy chain variable region sequence
EVQLVESGGGLVQPDRSLRLSCAASGFTFHDYAMHWVRQAPGKGLEWVSTISWNSGTIGYADSVKGRFTISRDNAKNSLYLQMNSLRAEDTALYYCAKDIQYGNYYYGMDVWGQGTTVTVSS

SEQ ID NO: 8 - protein, 107 aa, synthetic construct
  REGION 1..107: note = Light chain variable region sequence
EIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIK

(19) United States
(12) Patent Application Publication
(10) Pub. No.: US 2024/0409656 A1
(43) Pub. Date: Dec. 12, 2024

(54) METHODS OF TREATING CD20 EXPRESSING B-CELL CANCERS

(71) Applicant: GENMAB A/S, Valby (DK)

(72) Inventors: Nurgul KILAVUZ, Chatham, NJ (US); Mariana SACCHI, Madrid (ES)

(21) Appl. No.: 18/664,772

(22) Filed: May 15, 2024

Related U.S. Application Data
(60) Provisional application No. 63/569,470, filed on Mar. 25, 2024.
(60) Provisional application No. 63/467,370, filed on May 18, 2023.
(60) Provisional application No. 63/466,596, filed on May 15, 2023.

(51) Int. Cl. C07K 16/28; A61K 39/00

(52) U.S. Cl. CPC C07K 16/2887; C07K 16/2809; A61K 2039/505; A61K 2039/54; A61K 2039/545; A61K 2039/55511; C07K 2317/31

(57) ABSTRACT

The present invention relates to improved methods for reducing cytokine release syndrome following interruption of the epcoritamab dosing schedule for the treatment of CD20 B-cell expressing cancers.

### FIELD

The present invention relates to the use of Epcoritamab for the treatment of CD20 B-cell cancers when there has been a delay in the dosage schedule to minimize the release of cytokines following the resumption of dosing.

### BACKGROUND

A promising approach to improve targeted antibody therapy is by delivering cytotoxic cells specifically to the antigen-expressing cancer cells. This concept of using T-cells for efficient killing of tumor cells has been described in Staerz, et. al., 1985, Nature 314:628-631). However, initial clinical studies were rather disappointing mainly due to low efficacy, severe adverse effects (cytokine storm) and immunogenicity of the bispecific antibodies (Muller and Kontermann, 2010, BioDrugs 24:89-98). Advances in the design and application of bispecific antibodies have partially overcome the initial barrier of cytokine storm and improved clinical effectiveness without dose-limiting toxicities (Garber, 2014, Nat. Rev. Drug Discov. 13:799-801; Lum and Thakur, 2011, BioDrugs 25:365-379). Critical to overcome the initial barrier of cytokine storm as described for catumaxomab (Berek et al. 2014, Int. J. Gynecol. Cancer 24 (9): 1583-1589; Mau-Sørensen et al. 2015, Cancer Chemother. Pharmacol. 75:1065-1073) was the absence or silencing of the Fc domain.

The CD20 molecule (also called human B-lymphocyte-restricted differentiation antigen or Bp35) is a hydrophobic transmembrane protein with a molecular weight of approximately 35 kD located on pre-B and mature B lymphocytes (Valentine et al. (1989) J. Biol. Chem. 264 (19): 11282-11287; and Einfield et al., (1988) EMBO J. 7 (3): 711-717). CD20 is found on the surface of greater than 90% of B cells from peripheral blood or lymphoid organs and is expressed during early pre-B cell development and remains until plasma cell differentiation. CD20 is present on both normal B cells as well as malignant B cells. CD20 is expressed on greater than 90% of B cell non-Hodgkin's lymphomas (NHL) (Anderson et al. (1984) Blood 63 (6): 1424-1433), but is not found on hematopoietic stem cells, pro-B cells, normal plasma cells, or other normal tissues (Tedder et al. (1985) J. Immunol. 135 (2): 973-979).

Bispecific antibodies that bind to both CD3 and CD20 may be useful in therapeutic settings in which specific targeting and T cell-mediated killing of cells that express CD20 is desired.

CD3×CD20 bispecific antibodies have been described in the art, for example in Hutchings et al. (2021) Lancet 398:1157-1169; Gall et al. (2005) Experimental Hematology 33:452; Stanglmaier et al. (2008) Int. J. Cancer: 123, 1181; Wu et al. (2007) Nat Biotechnol. 25:1290-1297; Sun et al. (2015) Science Translational Medicine 7, 287ra70; U.S. Pat. No. 10,544,220; US 2021/0371538; WO2011014659; WO2011090762; WO2011028952; WO2014047231; WO 2016/110576; and WO 2021/224499. While advances have been made in reducing cytokine release syndrome following the initial dosing of CD3×CD20 bispecific antibodies, methods for minimizing cytokine release syndrome following a delay in the dosage schedule are needed.

### BRIEF DESCRIPTION OF DRAWINGS

FIG. 1: Example illustrating definition of safe re-priming window is defined based on the PopPK model after missing first full dose of EPKINLY/Epcoritamab. Horizontal dotted line marks the trough concentration at end of the priming dosing interval. Vertical dotted line marks the time it took for epcoritamab concentration to fall below the trough concentration after priming dose after missing the first full dose.

FIG. 2: Definition of safe re-priming window based on the rTTE model after missing first full dose of epcoritamab Abbreviations: CRS=cytokine release syndrome; rTTE=repeated time-to-event. Dash curve represents hazard over time for reference regimen where the first full dose was administered on schedule. Curve with asterisks represents hazard over time of a delayed regimen where the first full dose was delayed by 1 week. Curve with circle represents hazard over time of a delayed regimen where the first full dose was delayed by 2 weeks. Horizontal dotted line marks the peak instantaneous hazard from reference regimen.

FIG. 3: rTTE model-based simulation illustrating the length of safe re-priming window after a dose delay. Abbreviations: C1=cycle 1; rTTE=repeated time-to-event. Horizontal dash line marks the threshold where >95% of subjects could safely resume dosing after delay. Vertical dash lines (from left to right) represent switch safe re-priming window after missed intermediate/first full dose, missed second full dose, and missed full dose after Cycle 1, respectively.

FIG. 4: Simulated PK profiles—reference (nominal dosing schedule) compared to delayed dosing (with or without re-priming) (scenario 1). PI=prediction interval; PK=pharmacokinetic(s). Solid line represents median of simulated individual PK profile. Dash line represents geometric mean of simulated individual PK profile. Shaded represents 90% PI.

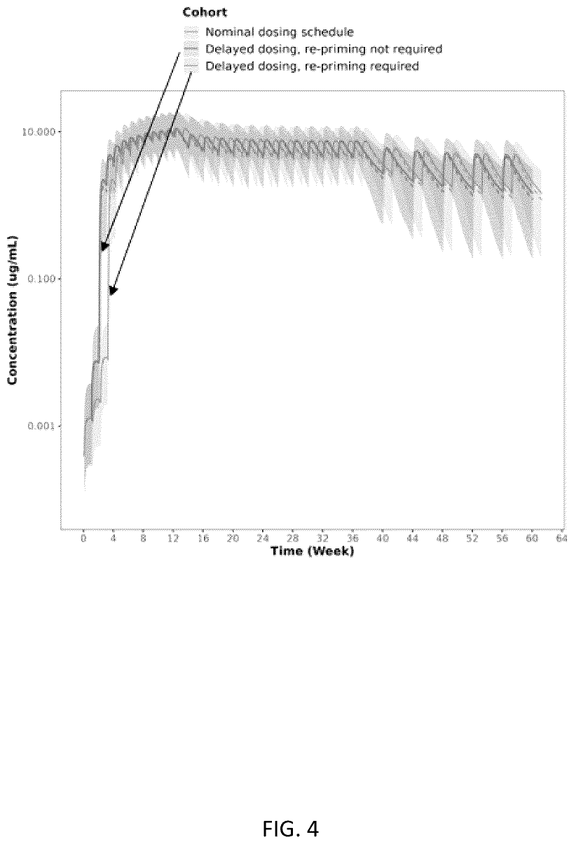

FIG. 5: Simulated PK profiles—reference (nominal dosing schedule) compared to delayed dosing (with or without re-priming) (scenario 2). PI=prediction interval; PK=pharmacokinetic(s). Solid line represents median of simulated individual PK profile. Dash line represents geometric mean of simulated individual PK profile. Shaded represents 90% PI.

FIG. 6: Simulated PK profiles—reference (nominal dosing schedule) compared to delayed dosing (with or without re-priming) (scenario 3). PI=prediction interval; PK=pharmacokinetic(s). Solid line represents median of simulated individual PK profile. Dash line represents geometric mean of simulated individual PK profile. Shaded represents 90% PI.3.

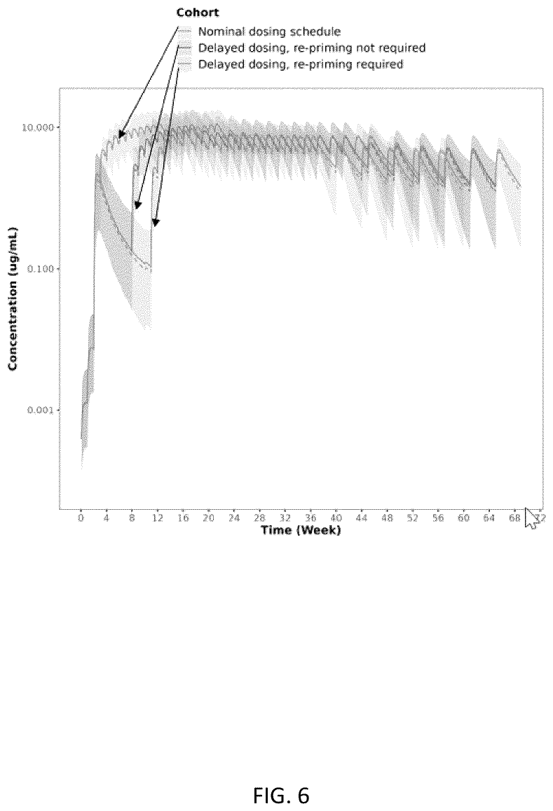

FIG. 7: Simulated PK profiles—reference (nominal dosing schedule) compared to delayed dosing (with or without re-priming) (scenario 4). PI=prediction interval; PK=pharmacokinetic(s). Solid line represents median of simulated individual PK profile. Dash line represents geometric mean of simulated individual PK profile. Shaded represents 90% PI.3.

FIG. 8: Simulated PK profiles—reference (nominal dosing schedule) compared to delayed dosing (with or without re-priming) (scenario 5). PI=prediction interval; PK=pharmacokinetic(s). Solid line represents median of simulated individual PK profile. Dash line represents geometric mean of simulated individual PK profile. Shaded represents 90% PI.3.

### SUMMARY

Provided herein are methods for treating patients with CD20 expressing cancers when the dosing schedule of EPKINLY is delayed due to adverse reactions or a missed dose. The resumption of the dosing schedule minimizes CRS by providing oral or intravenous corticosteroids, such as prednisolone or dexamethasone or an equivalent 30 to 120 minutes before restarting the first dose of EPKINLY as well as for 3 consecutive days following each of the 4 doses in the repriming cycle.

In the methods provided according to embodiments of the invention, a re-priming cycle is required if dosing of epcoritamab is delayed at certain timepoints as described below.
If an intermediate dose (0.8 mg dose) is delayed more than 1 day (ie, intermediate dose would occur more than 8 days after priming or any intermediate dose)
If the first full dose (48 mg dose) is delayed more than 7 days (ie, more than 14 days since the last intermediate dose)
For the second full dose (48 mg) onward, if the interval between the previous dose of epcoritamab and next epcoritamab dose exceeds 6 weeks.

Preferably, if restarting dosing with the priming or intermediate dose, the 4 days of consecutive corticosteroids must also be repeated for CRS prophylaxis until at least 1 full dose is re-administered within the appropriate dosing windows without subsequent occurrence of CRS grade ≥2. This applies to both Cycle 1 and to any re-priming within a re-priming cycle.

A re-priming cycle preferably consists of a weekly schedule of a priming dose (0.16 mg), an intermediate dose (0.8 mg), and 2 full doses (48 mg each). Premedication and prophylactic steroids should be given (similar to Cycle 1).

In one embodiment, a method for treating a CD20 expressing B-cell cancer in a human patient is described wherein said patient receives a 0.16 mg dose of epcoritamab on Cycle 1, Day 1 and the timing for the next scheduled dose is more than 8 days, dosing is resumed by: a) subcutaneously administering to the patient 0.16 mg of epcoritamab, b) subcutaneously administering to the patient 0.8 mg of epcoritamab the following week and, c) subcutaneously administering 48 mg of epcoritamab for two more weeks before starting Day 1 of the subsequent cycle.

In another embodiment, a method for treating a CD20 expressing B-cell cancer in a human patient is described wherein said patient receives a 0.8 mg dose of epcoritamab on Cycle 1, Day 8 and the time for the next scheduled dose of epcoritamab is 14 days or less, dosing is resumed with the dose that was missed and subsequent dosing is as scheduled wherein the 28-day dosing schedule is as follows:
a) Cycle 1, subcutaneously administering a dose of 48 mg at days 15 and 22;
b) Cycles 2 and 3, subcutaneously administering a dose of 48 mg on Days 1, 8, 15 and 22;
c) Cycles 4 to 9, subcutaneously administering a dose of 48 mg on Days 1 and 15 of cycles 4-9; and
d) subcutaneously administering a dose of 48 mg on Day 1 for all subsequent cycles.

In another embodiment, a method for treating a CD20 expressing B-cell cancer in a human patient is descried wherein said patient receives a 0.8 mg dose of epcoritamab on Cycle 1, Day 8 and the time for the next scheduled dose of epcoritamab is more than 14 days. Dosing is resumed by:
a. subcutaneously administering to the patient 0.16 mg of epcoritamab,
b. subcutaneously administering to the patient 0.8 mg of epcoritamab the following week, and,
c. subcutaneously administering two weekly doses of 48 mg of epcoritamab before starting Day 1 of the subsequent cycle.

In another embodiment, a method for treating a CD20 expressing B-cell cancer in a human patient is described wherein said patient receives a 48 mg dose of epcoritamab, and the time for the next scheduled dose of epcoritamab is 6 weeks or less. Dosing is resumed with the dose that was missed and subsequent dosing is based on a 28-day cycle as follows:
a) Cycle 1, subcutaneously administering a dose of 48 mg at days 15 and 22;
b) Cycles 2 and 3, subcutaneously administering a dose of 48 mg on Days 1, 8, 15 and 22;
c) Cycles 4 to 9, subcutaneously administering a dose of 48 mg on Days 1 and 15 of cycles 4-9; and
d) subcutaneously administering a dose of 48 mg on Day 1 for all subsequent cycles.

In another embodiment, a method for treating a CD20 expressing B-cell cancer in a human patient is described wherein said patient receives a 48 mg dose of epcoritamab, and the time for the next scheduled dose of epcoritamab is more than 6 weeks. Dosing is resumed with the dose that was missed and subsequent dosing is based on a 28-day dosing schedule is as follows:
a) Cycle 1, subcutaneously administering a dose of 48 mg at days 15 and 22;
b) Cycles 2 and 3, subcutaneously administering a dose of 48 mg on Days 1, 8, 15 and 22;
c) Cycles 4 to 9, subcutaneously administering a dose of 48 mg on Days 1 and 15 of cycles 4-9; and
d) subcutaneously administering a dose of 48 mg on Day 1 for all subsequent cycles.

### DETAILED DESCRIPTION

### Epcoritamab:

Epcoritamab, also referred to herein as EPINKLY, is a bispecific antibody recognizing the T-cell antigen CD3 and the B-cell antigen CD20. Epcoritamab triggers potent T-cell-mediated killing of CD20-expressing cells. The mechanism of action of epcoritamab is engagement of T-cells as effector cells to induce killing of CD20-expressing B-cells and tumor cells. This is a different mechanism of action compared to that of chemotherapy or conventional CD20-targeting monoclonal antibodies (mAbs) that can induce cytotoxicity through Fc-mediated effector functions such as antibody-dependent cellular cytotoxicity, antibody-dependent cell-mediated phagocytosis and complement-dependent cytotoxicity and in some cases programmed cell death.

Epcoritamab is generated using Genmab's DuoBody® technology (Labrijn et al., 2013; Labrijn et al., 2014). DuoBody molecules are bispecific antibodies with a regular IgG1 structure and biochemical characteristics typical of human IgG1. Accordingly, DuoBody molecules show normal binding to the neonatal Fc receptor (FcRn), resulting in the relatively long plasma half-life that is typical for IgG1 molecules. The Fc domain of epcoritamab has been modified to silence Fc-mediated effector functions, ensuring that epcoritamab does not activate T-cells through FcγR-mediated CD3 crosslinking. FcRn binding is preserved.

The term “Epcoritamab” or “EPKINLY™” refers to an IgG1 bispecific CD3×CD20 antibody comprising a first heavy and light chain pair as defined in SEQ ID NO: 1 and SEQ ID NO: 2, respectively, and comprising a second heavy and light chain pair as defined in SEQ ID NO: 3 and SEQ ID NO: 4. The first heavy and light chain pair comprises a region which binds to human CD3& (epsilon), the second heavy and light chain pair comprises a region which binds to human CD20. The first binding region comprises the VH and VL sequences as defined by SEQ ID NOs: 5 and 6, and the second binding region comprises the VH and VL sequences as defined by SEQ ID NOs: 7 and 8. This bispecific antibody can be prepared as described in WO 2016/110576.

As used herein, Epcoritamab is used to treat CD20 B-cell cancers. CD20 B-cell cancers refers to malignant lymphomas characterized by malignant transformation of the cells from lymphoid tissue. Historically, lymphomas have been divided into Hodgkin lymphoma and non-Hodgkin lymphoma (NHL). Malignant lymphoma originates from B-cells in >90% of the cases, less than 10% from T-cells and rarely from NK cells. The World Health Organization (WHO) has during the last two decades classified the many types of mature B-cell neoplasms including lymphomas and the most recent update has been in 2016 (Swerdlow et al., 2016). The majority of the mature B-cell neoplasms are considered to belong to the NHL. The prognosis of these malignancies is dependent on the type of lymphoma and the stage of the disease.

Clinically, NHL has been divided into indolent (slowly growing) lymphoma and aggressive (rapidly growing) lymphoma. The most common types of lymphoma are diffuse large B-cell lymphoma (DLBCL) which accounts for around 33% of NHL cases, follicular lymphoma (FL) representing 25% of NHL cases, and mantle cell lymphoma (MCL) 10% of NHL cases. Available clinical safety data from the ongoing EPCORE NHL-1 study (GCT3013-01; NCT03625037) shows that in 51% of the patients treated to date, Epcoritamab induces cytokine release syndrome (CRS) as a frequent adverse event (AE) (see Example 1). Typical cytokine release symptoms include chills, fever, and hypotension. To mitigate potential severe AEs from cytokine release in individual patients treated with epcoritamab several safety precautions can be implemented, including:
A priming dose, i.e., a lower dose than subsequent doses, as the first dose;
Premedication to mitigate CRS during the first 4 administrations (i.e., during Cycle 1), and optionally for later administrations. In particular embodiments, prophylactic corticosteroids, such as dexamethasone, prednisolone or equivalents thereof, are administered for 4 consecutive days (beginning pre-dose on the day of dosing) for the first 4 weekly administrations (i.e. during Cycle 1); for Cycle 2 and beyond, only if CRS ≥grade 2 occurs following the second full dose administration or the fourth administration of epcoritamab (C1D22) in Cycle 1 (or in any re-priming cycle); and,
Hospitalization is recommended for observation of patients during Cycle 1 (and in any re-priming cycle).

In some embodiments the dexamethasone equivalent is selected from the following:

| Steroid | Approximate Equivalent Dose | | |
|---|---|---|---|
| Betamethasone | 12 | mg | Long-acting |
| Dexamethasone | 15 | mg | Long-acting |
| Methylprednisolone | 80 | mg | Intermediate-acting |
| Triamcinolone | 80 | mg | Intermediate-acting |
| Prednisone | 100 | mg | Intermediate-acting |
| Prednisolone | 100 | mg | Intermediate-acting |
| Hydrocortisone | 400 | mg | Short acting |
| Cortisone | 500 | mg | Short-acting |

Preferably, equivalent is dosed at the Approximate Equivalent Dose provided in the table.

### Indications:

In some embodiments, EPKINLY can be used to treat patients with relapsed or refractory diffuse large B-cell lymphoma (DLBCL), not otherwise specified, including DLBCL arising from indolent lymphoma, and high-grade B cell lymphoma after two or more lines of systemic therapy.

In other embodiments, Epkinly can be used to treat patients with relapsed or refractory large B-cell lymphoma (LBCL) after two or more lines of systemic therapy, including diffuse large B-cell lymphoma (DLBCL); including those transformed from indolent lymphomas, high-grade B-cell lymphoma (HGBCL), primary mediastinal large B-cell lymphoma (PMBCL), and follicular lymphoma grade 3B (FL Gr 3B).

In yet other embodiments, EPKINLY can be used to treat relapsed or refractory large B-cell lymphoma, CD20 positive relapsed or refractory diffuse large B-cell lymphoma after two or more lines of systemic therapy and relapsed or refractory follicular lymphoma.

### Dosing and Premedication Schedules:

The subcutaneous dosage schedule for EPKINLY is provided in Table 1. As shown in Table 1, EPKINLY is administered in 28-day cycles until disease progression or unacceptable toxicity.

TABLE 1
EPKINLY Dosage Schedule

| Cycle of treatment^{a} | Day of treatment | Dose of EPKINLY | | |
|---|---|---|---|---|
| Cycle 1 | 1 | Step-up dose 1 | 0.16 | mg |
| | 8 | Step-up dose 2 | 0.8 | mg |
| | 15 | First full dose | 48 | mg |

| | | |
|---|---|---|
| | 22 | 48 mg |
| Cycles 2 and 3 | 1, 8, 15 and 22 | 48 mg |
| Cycles 4 to 9 | 1 and 15 | 48 mg |
| Cycle 10 and beyond | 1 | 48 mg |

^{a}Cycle = 28 days

During Cycle 1, the premedications shown in Table 2 are administered to reduce the risk of cytokine release syndrome (CRS). In some embodiments, these premedications can be administered in subsequent cycles if the patient is at risk for CRS.

TABLE 2
EPKINLY Premedication and CRS Prophylaxis

| | Patients | | |
|---|---|---|---|
| | requiring | | |
| Cycle | premedication | Premedication | Administration |
| Cycle 1 | All patients | Prednisolone (100 mg oral | 30-120 minutes prior to |
| | | or intravenous) or | each weekly |
| | | Dexamethasone (15 mg | administration of |
| | | oral or intravenous) or | EPKINLY |
| | | equivalent | And for three consecutive |
| | | | days following each |
| | | | weekly administration of |
| | | | EPKINLY in Cycle 1 |
| | | Diphenhydramine (50 mg | 30-120 minutes prior to |
| | | oral or intravenous) or | each weekly |
| | | equivalent | administration of |
| | | Acetaminophen (650 to | EPKINLY |
| | | 1,000 mg oral) | |
| Cycle 2+ | Patients who | Prednisolone (100 mg oral | 30-120 minutes prior to |
| | experienced | or intravenous) or | next administration of |
| | Grade 2 or 3^{a} | Dexamethasone (15 mg | EPKINLY after a Grade 2 |
| | CRS with | oral or intravenous) or | or 3^{a} CRS event |
| | previous dose | equivalent | And for three consecutive |
| | | | days following the next |
| | | | administration of |
| | | | EPKINLY until |
| | | | EPKINLY is given |
| | | | without subsequent CRS |
| | | | of Grade 2 or higher |

^{a}Patients will be permanently discontinued from EPKINLY after Grade 4 CRS.

In some embodiments, an epcoritamab dosing cycle can be delayed due to the management of adverse events, or because a patient missed the next dose in a cycle. When a delay in dosing occurs, Epcoritamab dosing can be restarted as described in Table 3.

TABLE 3
Restarting Therapy with EPKINLY After Dosage Delay

| Last Dose | Time Since the Last | |
|---|---|---|
| Administered | Dose Administered | Action for Next Dose(s)^{a} |
| 0.16 mg on Cycle | More than 8 days | Repeat 0.16 mg, then administer 0.8 mg the |
| 1 Day 1 | | following week, followed by two weekly doses |
| | | of 48 mg. Then resume the planned dosage |
| | | schedule beginning with Day 1 of the |
| | | subsequent cycle. |
| 0.8 mg on Cycle | 14 days or less | Administer 48 mg then resume the |
| 1 Day 8 | | recommended dosage schedule. |
| | More than 14 days | Repeat 0.16 mg, then administer 0.8 mg the |
| | | following week, followed by two weekly doses |
| | | of 48 mg. Then resume the planned dosage |
| | | schedule beginning with Day 1 of the |
| | | subsequent cycle. |
| 48 mg on Cycle 1 | 6 weeks or less | Administer 48 mg, then resume the |
| Day 15 onwards | | recommended dosage schedule. |
| | More than 6 weeks | Repeat 0.16 mg, then administer 0.8 mg the |
| | | following week, followed by two weekly doses |
| | | of 48mg. Then resume the planned dosage |
| | | schedule beginning with Day 1 of the |
| | | subsequent cycle. |

^{a}Administer pretreatment medication prior to EPKINLY dose and monitor patients accordingly.

### Dosage Modifications and Management of Adverse Reactions:

### Cytokine Release Syndrome (CRS)

If CRS is diagnosed or suspected, withhold EPKINLY until CRS resolves. Manage as described in Table 4 and consider further management per current practice guidelines. Administer supportive therapy for CRS, which may include intensive care for severe or life-threatening CRS.

TABLE 4
Management of Cytokine Release Syndrome

| Grade^{a} | Presenting Symptoms | Actions |
|---|---|---|
| Grade 1 | Temperature ≥100.4° F. (38° C.)^{b} | Withhold EPKINLY and manage |
| | | per current practice guidelines. |
| | | Ensure CRS symptoms are resolved |
| | | prior to next dose of EPKINLY.^{c} |
| Grade 2 | Temperature ≥100.4° F. (38° C.)^{b} with: | Withhold EPKINLY and manage |
| | Hypotension not requiring vasopressors | per current practice guidelines. |
| | and/or | Ensure CRS symptoms are resolved |
| | Hypoxia requiring low-flow oxygene by | prior to next dose of EPKINLY.^{c} |
| | nasal cannula or blow-by. | Administer premedicationd prior to |
| | | next dose of EPKINLY. |
| | | For the next dose of EPKINLY, |
| | | monitor more frequently and |
| | | consider hospitalization. |
| Grade 3 | Temperature ≥100.4° F. (38° C.)^{b} with: | Withhold EPKINLY and manage |
| | Hypotension requiring a vasopressor | per current practice guidelines, |
| | (with or without vasopressin) | which may include intensive care. |
| | and/or | Ensure CRS symptoms are resolved |
| | Hypoxia requiring high-flow oxygen^{e} | prior to the next dose of |
| | by nasal cannula, face mask, non- | EPKINLY.^{c} |
| | rebreather mask, or Venturi mask. | Administer premedication^{d} prior to |
| | | next dose of EPKINLY. |
| | | Hospitalize for the next dose of |
| | | EPKINLY. |
| | | Recurrent Grade 3 CRS |
| | | Permanently discontinue |
| | | EPKINLY. |
| | | Manage CRS per current practice |
| | | guidelines and provide supportive |
| | | therapy, which may include |
| | | intensive care. |
| Grade 4 | Temperature ≥100.4° F. (38° C.)^{b} with: | Permanently discontinue |
| | Hypotension requiring multiple | EPKINLY. |
| | vasopressors (excluding vasopressin) | Manage CRS per current practice |
| | and/or | guidelines and provide supportive |
| | Hypoxia requiring oxygen by positive | therapy, which may include |
| | pressure (e.g., CPAP, BiPAP, | intensive care. |
| | intubation and mechanical ventilation). | |

^{a}Based on American Society for Transplantation and Cellular Therapy (ASTCT) 2019 grading for CRS.
^{b}Premedication may mask fever, therefore if clinical presentation is consistent with CRS, follow these management guidelines.
^{c}Refer to Table 2 for information on restarting EPKINLY after dose delays.
^{d}If Grade 2 or 3 CRS occurs with the second full dose (48 mg) or beyond, administer CRS premedications with each subsequent dose until a EPKINLY dose is given without subsequent CRS of Grade 2 or higher. Refer to Table 3 for additional information on premedication.
^{e}Low-flow oxygen defined as oxygen delivered at <6 L/minute; high-flow oxygen defined as oxygen delivered at ≥6 L/minute.

### Immune Effector Cell-Associated Neurological Toxicity Syndrome (ICANS)

Patients should be monitored for signs and symptoms of ICANS. At the first sign of ICANS withhold EPKINLY and conduct a neurology evaluation to rule out other causes of neurologic symptoms. Provide supportive therapy, which may include intensive care. ICANS can be managed as described in Table 5 and current practice guidelines.

TABLE 5
Management of Immune Effector Cell-Associated Neurotoxicity Syndrome

| Grade^{a} | Presenting Symptoms^{b} | Actions |
|---|---|---|
| Grade 1 | ICE score 7-9^{c}, | Withhold EPKINLY until ICANS |
| | Or depressed level of | resolves.^{e} |
| | consciousness^{d}: awakens | Monitor neurologic symptoms and |
| | spontaneously. | consider consultation with neurologist and |
| | | other specialists for further evaluation and |
| | | management, including consideration for |
| | | starting non-sedating, anti-seizure |
| | | medicines for seizure prophylaxis. |
| Grade 2 | ICE score 3-6^{c}, | Withhold EPKINLY until ICANS |
| | Or depressed level of | resolves.^{e} |
| | consciousness^{d}: awakens to voice. | Administer dexamethasonef 10 mg |
| | | intravenously every 6 hours. Continue |
| | | dexamethasone use until resolution to |
| | | Grade 1 or less, then taper. |
| | | Monitor neurologic symptoms and |
| | | consider consultation with neurologist and |
| | | other specialists for further evaluation and |
| | | management, including consideration for |
| | | starting non-sedating, anti-seizure |
| | | medicines for seizure prophylaxis. |
| Grade 3 | ICE score 0-2^{c}, | First Occurrence of Grade 3 ICANS |
| | Or depressed level of | Withhold EPKINLY until ICANS |
| | consciousness^{d}: awakens only to | resolves.^{e} |
| | tactile stimulus, | Administer dexamethasone^{f} 10 mg |
| | Or seizures,^{d} either: | intravenously every 6 hours. Continue |
| | Any clinical seizure, focal or | dexamethasone use until resolution to |
| | generalized, that resolves | Grade 1 or less, then taper. |
| | rapidly, or | Monitor neurologic symptoms and |
| | Non-convulsive seizures on | consider consultation with neurologist and |
| | electroencephalogram (EEG) | other specialists for further evaluation and |
| | that resolve with intervention, | management, including consideration for |
| | Or raised intracranial pressure: | starting non-sedating, anti-seizure |
| | focal/local edema on | medicines for seizure prophylaxis. |
| | neuroimaging.^{d} | Provide supportive therapy, which may |
| | | include intensive care. |
| | | Recurrent Grade 3 ICANS |
| | | Permanently discontinue EPKINLY |
| | | Administer dexamethasone^{f} 10 mg |
| | | intravenously every 6 hours. Continue |
| | | dexamethasone use until resolution to |
| | | Grade 1 or less, then taper. |
| | | Monitor neurologic symptoms and |
| | | consider consultation with neurologist and |
| | | other specialists for further evaluation and |
| | | management, including consideration for |
| | | starting non-sedating, anti-seizure |
| | | medicines for seizure prophylaxis. |
| | | Provide supportive therapy, which may |
| | | include intensive care. |
| Grade 4 | ICE score 0^{c}, | Permanently discontinue EPKINLY. |
| | Or depressed level of | Administer dexamethasonef 10 mg |
| | consciousness^{d}: either: | intravenously every 6 hours. Continue |
| | Patient is unarousable or | dexamethasone use until resolution to |
| | requires vigorous or repetitive | Grade 1 or less, then taper. |
| | tactile stimuli to arouse, or | Alternatively, consider administration of |
| | Stupor or coma | methylprednisolone 1,000 mg per day |
| | Or seizures,^{d} either: | intravenously and continue |
| | Life-threatening prolonged | methylprednisolone 1,000 mg per day |
| | seizure (>5 minutes), or | intravenously for 2 or more days. |
| | Repetitive clinical or electrical | Monitor neurologic symptoms and |
| | seizures without return to | consider consultation with neurologist and |
| | baseline in between, | other specialists for further evaluation and |
| | Or motor findings^{d}: | management, including consideration for |
| | Deep focal motor weakness, | starting non-sedating, anti-seizure |
| | such as hemiparesis or | medicines for seizure prophylaxis. |
| | paraparesis, | Provide supportive therapy, which may |
| | or raised intracranial | include intensive care. |
| | pressure/cerebral edema,^{d} with | |
| | signs/symptoms such as: | |
| | Diffuse cerebral edema on | |
| | neuroimaging, or | |
| | Decerebrate or decorticate | |
| | posturing, or | |
| | Cranial nerve VI palsy, or | |
| | Papilledema, or | |
| | Cushing's triad. | |

^{a}Based on American Society for Transplantation and Cellular Therapy (ASTCT) 2019 grading for ICANS.
^{b}Management is determined by the most severe event, not attributable to any other cause.
^{c}If patient is arousable and able to perform Immune Effector Cell-Associated Encephalopathy (ICE) Assessment, assess: Orientation (oriented to year, month, city, hospital = 4 points); Naming (names 3 objects, e.g., point to clock, pen, button = 3 points); Following Commands (e.g., “show me 2 fingers” or “close your eyes and stick out your tongue” = 1 point); Writing (ability to write a standard sentence = 1 point); and Attention (count backwards from 100 by ten = 1 point). If patient is unarousable and unable to perform ICE Assessment (Grade 4 ICANS) = 0 points.
^{d}Not attributable to any other cause.
^{e}See Table 2 for recommendations on restarting EPKINLY after dose delays [see Dosage and Administration (2.3)]
^{f}All references to dexamethasone administration are dexamethasone or equivalent.

### Other Adverse Reactions:

In some embodiments, prophylactic antibiotic, antiviral and antifungal therapies are recommended for patients who are at an increased risk for these infections. For example, for patients with a history of recurrent herpes virus infections, herpes infection during previous anti-lymphoma therapy, neutropenia and/or low CD4+ cell counts (<200 cells/μL) prophylactic antiviral therapy is mandatory, e.g., acyclovir 400 mg three times a day orally.

By way of another example, prophylaxis against Pneumocystis jirovecii, e.g., oral trimethoprim/sulfamethoxazole 160 mg/800 mg every other day is mandatory when 4 or more consecutive days of corticosteroids are given (eg, during CRS prophylaxis or adverse event (AE) management), as well as for patients who are considered at increased risk, e.g., patients with low CD4+ cell counts (<350 cells/μL).

In other embodiments, complete blood counts are used throughout treatment to monitor for cytopenias. Based on the severity of cytopenias, temporarily withhold or permanently discontinue EPKINLY. Prophylactic granulocyte colony-stimulating factor administration should be used as applicable.

EPKINLY dosage modifications for other adverse reactions are described in Table 6.

TABLE 6
Dosage Modifications for Other Adverse Reactions

| Adverse Reaction¹ | Severity¹ | Action |
|---|---|---|
| Infections | Grades 1-4 | Withhold EPKINLY in |
| | | patients with active |
| | | infection, until the |
| | | infection resolves.² |
| | | For Grade 4, consider |
| | | permanent |
| | | discontinuation of |
| | | EPKINLY. |
| Neutropenia | Absolute neutrophil count | Withhold EPKINLY until |
| | less than 0.5 × 10⁹/L | absolute neutrophil count |
| | | is 0.5 × 10⁹/L or higher.² |
| Thrombocytopenia | Platelet count less than | Withhold EPKINLY until |
| | 50 × 10⁹/L | platelet count is 50 × |
| | | 10⁹/L or higher.² |
| Other Adverse Reactions | Grade 3 or higher | Withhold EPKINLY until |
| | | the toxicity resolves to |
| | | Grade 1 or baseline.² |

¹Based on National Cancer Institute Common Terminology Criteria for Adverse Events (NCI CTCAE), version 5.0.
²See Table 2 for restarting EPKINLY after dosage delays..

In a preferred embodiment, epcoritamab is provided for subcutaneous use in a sterile, preservative-free, clear to slightly opalescent, colorless to slightly yellow solution, free of visible particles, solution. For the priming doses, epcoritamab is provided as single-dose 4 mg/0.8 mL vial contains epcoritamab (4 mg), acetic acid (0.19 mg), polysorbate 80 (0.32 mg), sodium acetate (1.7 mg), sorbitol (21.9 mg) and Water for Injection, USP. The pH is 5.5.

For the full dose cycles, epcoritamab is provided as a single-dose 48 mg/0.8 mL vial containing epcoritamab (48 mg), acetic acid (0.19 mg), polysorbate 80 (0.32 mg), sodium acetate (1.7 mg), sorbitol (21.9 mg) and Water for Injection, USP. The pH is 5.5.

### EXAMPLES

### Example 1: Safety Findings from EPCORE NHL-1 Study (GCT3013-01; NCT03625037)

### Cytokine Release Syndrome:

Cytokine release syndrome occurred in 51% of patients receiving EPKINLY at the recommended dose in the clinical trial, with Grade 1 CRS occurring in 37%, Grade 2 in 17%, and Grade 3 in 2.5% of patients. Recurrent CRS occurred in 16% of patients. Of all the CRS events, most (92%) occurred during Cycle 1. In Cycle 1, 9% of CRS events occurred after the 0.16 mg dose on Cycle 1 Day 1, 16% after the 0.8 mg dose on Cycle 1 Day 8, 61% after the 48 mg dose on Cycle 1 Day 15, and 6% after the 48 mg dose on Cycle 1 Day 22.

The median time to onset of CRS from the most recent administered EPKINLY dose across all doses was 24 hours (range: 0 to 10 days). The median time to onset after the first full 48 mg dose was 21 hours (range: 0 to 7 days). CRS resolved in 98% of patients and the median duration of CRS events was 2 days (range: 1 to 27).

In patients who experienced CRS, the signs and symptoms included pyrexia, hypotension, hypoxia, dyspnea, chills, and tachycardia. Concurrent neurological adverse reactions associated with CRS occurred in 2.5% of patients and included headache, confusional state, tremors, dizziness, and ataxia.

Initiate therapy according to EPKINLY dosing schedule (see Table 1). Administer pretreatment medications to reduce the risk of CRS and monitor patients for potential CRS following EPKINLY accordingly (see Table 2). Following administration of the first 48 mg dose, patients should be hospitalized for 24 hours. At the first signs or symptoms of CRS, immediately evaluate patients for hospitalization, manage per current practice guidelines, and administer supportive care as appropriate. Withhold or discontinue EPKINLY based on the severity of CRS (see Table 4).

### Immune Effector Cell-Associated Neurotoxicity Syndrome (ICANS)

Immune Effector Cell-Associated Neurotoxicity Syndrome occurred in 6% (10/157) of patients receiving EPKINLY at the recommended dose in the clinical trial, with Grade 1 ICANS in 4.5% and Grade 2 ICANS in 1.3% of patients. There was one (0.6%) fatal ICANS occurrence. Of the 10 ICANS events, 9 occurred within Cycle 1 of EPKINLY treatment, with a median time to onset of ICANS of 16.5 days (range: 8 to 141 days) from the start of treatment. Relative to the most recent administration of EPKINLY, the median time to onset of ICANS was 3 days (range: 1 to 13 days). The median duration of ICANS was 4 days (range: 0-8 days) with ICANS resolving in 90% of patients with supportive care. Clinical manifestations of ICANS included, but were not limited to, confusional state, lethargy, tremor, dysgraphia, aphasia, and non-convulsive status epilepticus. The onset of ICANS can be concurrent with CRS, following resolution of CRS, or in the absence of CRS.

At the first signs or symptoms of ICANS, immediately evaluate the patient and provide supportive therapy based on severity. Withhold or discontinue EPKINLY per Table 5 and consider further management per current practice guidelines.

### Infections

In the clinical trial, serious infections, including opportunistic infections, were reported in 15% of patients treated with EPKINLY at the recommended dose with Grade 3 or 4 infections in 14% and fatal infections in 1.3%. The most common Grade 3 or greater infections were sepsis, COVID-19, urinary tract infection, pneumonia, and upper respiratory tract infection. Infections can be managed as described in Table 6.

### Cytopenias

Among patients who received the recommended dosage in the clinical trial, Grade 3 or 4 decreased neutrophils occurred in 32%, decreased hemoglobin in 12%, and decreased platelets in 12% of patients. Febrile neutropenia occurred in 2.5%. Cytopenias can be managed as described in Table 6.

### Drug Interactions

For certain CYP substrates, minimal changes in the concentration may lead to serious adverse reactions. Monitor for toxicity or drug concentrations of such CYP substrates when co-administered with EPKINLY.

Epcoritamab causes release of cytokines (see Example 3, Clinical Pharmacology) that may suppress activity of CYP enzymes, resulting in increased exposure of CYP substrates. Increased exposure of CYP substrates is more likely to occur after the first dose of EPKINLY on Cycle 1 Day 1 and up to 14 days after the first 48 mg dose on Cycle 1 Day 15 and during and after CRS.

### Example 2: Clinical Trial Experience

The safety of EPKINLY was evaluated in EPCORE NHL-1, a single-arm study of patients with relapsed or refractory LBCL after two or more lines of systemic therapy, including DLBCL not otherwise specified, DLBCL arising from indolent lymphoma, high grade B-cell lymphoma, and other B-cell lymphomas. A total of 157 patients received EPKINLY via subcutaneous injection until disease progression or unacceptable toxicities according to the following 28-day cycle schedule:
Cycle 1: EPKINLY 0.16 mg on Day 1, 0.8 mg on Day 8, 48 mg on Days 15 and Day 22
Cycles 2-3: EPKINLY 48 mg on Days 1, 8, 15, and 22
Cycles 4-9: EPKINLY 48 mg on Days 1 and 15
Cycles 10 and beyond: EPKINLY 48 mg on Day 1

Of the 157 patients treated, the median age was 64 years (range: 20 to 83), 60% male, and 97% had an ECOG performance status of 0 or 1. Race was reported in 133 (85%) patients; of these patients, 61% were White, 19% were Asian, and 0.6% were Native Hawaiian or Other Pacific Islander. There were no Black or African American or Hispanic or Latino patients treated in the clinical trial as reported. The median number of prior therapies was 3 (range: 2 to 11). The study excluded patients with CNS involvement of lymphoma, allogeneic HSCT or solid organ transplant, an ongoing active infection, and any patients with known impaired T-cell immunity.

The median duration of exposure for patients receiving EPKINLY was 5 cycles (range: 1 to 20 cycles).

Serious adverse reactions occurred in 54% of patients who received EPKINLY. Serious adverse reactions in ≥2% of patients included CRS, infections (including sepsis, COVID-19, pneumonia and upper respiratory tract infections), pleural effusion, febrile neutropenia, fever, and ICANS. Fatal adverse reactions occurred in 3.8% of patients who received EPKINLY, including COVID-19 (1.3%), hepatotoxicity (0.6%), ICANS (0.6%), myocardial infarction (0.6%), and pulmonary embolism (0.6%).

Permanent discontinuation of EPKINLY due to an adverse reaction occurred in 3.8% of patients. Adverse reactions which resulted in permanent discontinuation of EPKINLY included COVID-19, CRS, ICANS, pleural effusion, and fatigue.

Dosage interruptions of EPKINLY due to an adverse reaction occurred in 34% of patients who received EPKINLY. Adverse reactions which required dosage interruption in ≥3% of patients included CRS, neutropenia, sepsis and thrombocytopenia.

The most common (≥20%) adverse reactions were CRS, fatigue, musculoskeletal pain, injection site reactions, pyrexia, abdominal pain, nausea, and diarrhea. The most common Grade 3 to 4 laboratory abnormalities (>10%) were decreased lymphocyte count, decreased neutrophil count, decreased white blood cell count, decreased hemoglobin, and decreased platelets.

Table 7 summarizes the adverse reactions in EPCORE NHL-1.

TABLE 7
Adverse Reactions (≥10%) in Patients with Relapsed
or Refractory LBCL Who Received EPKINLY in EPCORE NHL-1

EPKINLY
(N = 157)

| | | |
|---|---|---|
| | All Grades | Grade 3 or 4 |
| Adverse Reaction | (%) | (%) |

Immune system disorders

| | | |
|---|---|---|
| Cytokine release syndrome* | 51 | 2.5^{#} |

General disorders and administration site conditions

| | | |
|---|---|---|
| Fatigue^{a} | 29 | 2.5^{#} |
| Injection site reactions^{b} | 27 | 0 |
| Pyrexia | 24 | 0 |
| Edema^{c} | 14 | 1.9^{#} |

Musculoskeletal and connective tissue disorders

| | | |
|---|---|---|
| Musculoskeletal pain^{d} | 28 | 1.3^{#} |

Gastrointestinal disorders

| | | |
|---|---|---|
| Abdominal pain^{e} | 23 | 1.9^{#} |
| Diarrhea | 20 | 0 |
| Nausea | 20 | 1.3^{#} |
| Vomiting | 12 | 0.6^{#} |

Skin and subcutaneous disorders

| | | |
|---|---|---|
| Rash^{f} | 15 | 0.6^{#} |

Nervous system disorder

| | | |
|---|---|---|
| Headache | 13 | 0.6^{#} |

Metabolism and nutrition disorders

| | | |
|---|---|---|
| Decreased appetite | 12 | 0.6^{#} |

Cardiac disorders

| | | |
|---|---|---|
| Cardiac arrhythmias^{g} | 10 | 0.6^{#} |

^{#}Only includes grade 3 adverse reactions.
*CRS was graded using ASTCT consensus criteria (Lee et al., 2019).
^{a}Fatigue includes asthenia, fatigue, lethargy.
^{b}Injection site reactions includes injection site erythema, injection site hypertrophy, injection site inflammation, injection site mass, injection site pain, injection site pruritus, injection site rash, injection site reaction, injection site swelling, injection site urticaria.
^{c}Edema includes edema, edema peripheral, face edema, generalized edema, peripheral swelling.
^{d}Musculoskeletal pain includes back pain, bone pain, flank pain, musculoskeletal chest pain, musculoskeletal pain, myalgia, neck pain, non-cardiac chest pain, pain, pain in extremity, spinal pain
^{e}Abdominal pain includes abdominal discomfort, abdominal pain, abdominal pain lower, abdominal pain upper, abdominal tenderness.
^{f}Rash includes dermatitis bullous, erythema, palmar erythema, penile erythema, rash, rash erythematous, rash maculo-papular, rash pustular, recall phenomenon, seborrheic dermatitis, skin exfoliation.
^{g}Cardiac arrhythmias includes bradycardia, sinus bradycardia, sinus tachycardia, supraventricular extrasystoles, supraventricular tachycardia, tachycardia.

Clinically relevant adverse reactions in <10% of patients who received EPKINLY included ICANS, sepsis, pleural effusion, COVID-19, pneumonia (including pneumonia and COVID-19 pneumonia), tumor flare, febrile neutropenia, upper respiratory tract infections, and tumor lysis syndrome.

Table 8 summarizes laboratory abnormalities in EPCORE NHL-1.

TABLE 8
Select Laboratory Abnormalities (≥20%) That Worsened
from Baseline in Patients with Relapsed or Refractory
LBCL Who Received EPKINLY in EPCORE NHL-1

| | EPKINLY¹ | |
|---|---|---|
| Laboratory Abnormality | All Grades (%) | Grade 3 or 4 (%) |

Hematology

| | | |
|---|---|---|
| Lymphocyte count decreased | 87 | 77 |
| Hemoglobin decreased | 62 | 12 |
| White blood cells decreased | 53 | 22 |
| Neutrophils decreased | 50 | 32 |
| Platelets decreased | 48 | 12 |

Chemistry

| | | |
|---|---|---|
| Sodium decreased | 56 | 2.6 |
| Phosphate decreased² | 56 | N/A |
| Aspartate aminotransferase increased | 48 | 4.6 |
| Alanine aminotransferase increased | 45 | 5.3 |
| Potassium decreased | 34 | 5.3 |
| Magnesium decreased | 31 | 0 |
| Creatinine increased | 24 | 3.3 |
| Potassium increased | 21 | 1.3 |

Laboratory abnormalities were graded based on CTCAE version 5.0.
¹The denominator used to calculate the rate varied from 146 to 153 based on the number of patients with a baseline value and at least one post-treatment value.
²CTCAE version 5.0 does not include numeric thresholds for grading of hypophosphatemia; All grades represent patients with lab value <LLN.

The efficacy population included 148 patients with DLBCL, not otherwise specified (NOS), including DLBCL arising from indolent lymphoma, and high-grade B-cell lymphoma. Of the 148 patients, the median age was 65 years (range: 22 to 83), 62% were male, 97% had an ECOG performance status of 0 or 1, and 3% had an ECOG performance status of 2. Race was reported in 125 (84%) patients; of these patients, 61% were White, 20% were Asian, and 0.7% were Native Hawaiian or Other Pacific Islander. There were no Black or African American or Hispanic or Latino patients treated in the clinical trial as reported. The diagnosis was DLBCL NOS in 86%, including 27% with DLBCL transformed from indolent lymphoma, and high-grade B-cell lymphoma in 14%. The median number of prior therapies was 3 (range: 2 to 11), with 30% receiving 2 prior therapies, 30% receiving 3 prior therapies, and 40% receiving 4 or more prior therapies. Eighteen percent had prior autologous HSCT, and 39% had prior chimeric antigen receptor (CAR) T-cell therapy. Eighty-two percent of patients had disease refractory to last therapy and 29% of patients were refractory to CAR T-cell therapy.

Efficacy was established based on overall response rate (ORR) determined by Lugano 2014 criteria as assessed by Independent Review Committee (IRC) and duration of response. The efficacy results are summarized in Table 10.

TABLE 9
Efficacy Results in EPCORE NHL-1 in Patients
with DLBCL and High-grade B-cell Lymphoma

| | EPKINLY | |
|---|---|---|
| Endpoint^{a} | (N = 148) | |
| ORR, n (%) | 90 | (61) |
| (95% CI) | (52.5, 68.7) | |
| CR, n (%) | 56 | (38) |
| (95% CI) | (30.0, 46.2) | |
| PR, n (%) | 34 | (23) |
| (95% CI) | (16.5, 30.6) | |
| DOR | | |
| Median (95% CI), months | 15.6 | (9.7, NR) |
| 9-month estimateb % (95% CI) | 63 | (51.5, 72.4) |

ORR = overall response rate; CI = confidence interval; CR = complete response; PR = partial response; DOR = duration of response; NR = not reached.
^{a}Determined by Lugano criteria (2014) as assessed by independent review committee (IRC).
^{b}Kaplan-Meier estimate.

The median time to response was 1.4 months (range: 1 to 8.4 months). Among responders, the median follow-up for DOR was 9.8 months (range: 0.0 to 17.3 months).

### Example 3: Clinical Pharmacology

### Pharmacodynamics:

### Circulating B Cell Count

Circulating B cells decreased to undetectable levels (<10 cells/microliter) after administration of the approved recommended dosage of EPKINLY in patients who had detectable B cells at treatment initiation by Cycle 1 Day 15 (after the first full dose of 48 mg) and the depletion was sustained while patients remained on treatment.

### Cytokine Concentrations

Plasma concentrations of cytokines (IL-2, IL-6, IL-10, TNF-α, and IFN-γ) were measured. Transient elevation of circulating cytokines was observed at dose levels of 0.04 mg and above. After administration of the approved recommended dosage of EPKINLY, the cytokine levels increased within 24 hours after the first dose on Cycle 1 Day 1, reached maximum levels after the first 48 mg dose on Cycle 1 Day 15, and returned to baseline prior to the next 48 mg full dose on Cycle 1 Day 22.

### Example 4: Defining Safe Re-Priming Windows

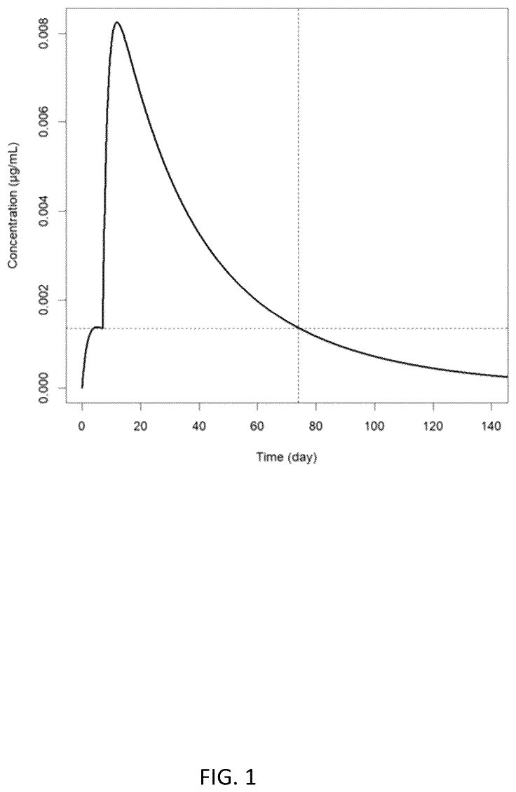

The re-priming recommendations were based on population PK (PopPK) modeling and are supported by the observed clinical data. The PopPK model-based approach assumed that re-priming was required when EPKINLY concentrations dropped below the trough concentration (C_{trough}) following the first priming dose (see FIG. 1). An individual subjects's EPKINLY concentration profile was simulated using post hoc individual PK parameters after missing the scheduled EPKINLY first full dose, second full dose, or missing the full dose after Cycle 1. The time it took for each subject's EPKINLY concentration to fall below C_{trough} after the priming dose was defined as the safe re-priming window. The calculated safe re-priming window from all subjects was summarized. After missing a scheduled first full dose, the safe re-priming window (5th percentile; 95% coverage and only 5% subjects with concentrations below their corresponding C_{trough} after priming dose) was 4.76 weeks. After missing a scheduled second full dose, the safe repriming window (5th percentile) was 18.4 weeks. After missing a scheduled full dose after Cycle 1, the safe re-priming window (5th percentile) was 25.1 weeks. The re-priming time window after missing the intermediate dose could not be calculated using the population PK-based approach because the safe repriming concentration threshold was defined as C_{trough} after the priming dose. Thus, any delay in the intermediate dose would result in EPKINLY concentrations falling below the defined threshold. As such, a conservative re-priming strategy was proposed for a 1-day delay of the intermediate dose (i.e., >8 days between the priming dose (0.16 mg) and intermediate dose (0.8 mg). Similarly, a repeated time-to-event (rTTE) modeling approach was explored and provided similar results (see below).

The more conservative values from the popPK model-based simulations were used to identify the re-priming windows. For delayed intermediate dose, a repriming window of 1 day (8 days after priming dose) was selected. For delayed first full dose, a repriming window of 7 days (14 days after intermediate dose) was selected. For the delayed second full dose and full doses after Cycle 1, a conservative 6-week window were chosen.

To further support these re-priming windows, simulations of individual predicted PK profiles of EPKINLY were performed. These are described below:
Reference (nominal dosing schedule)
   Cohort 1 (nominal dosing schedule): Post hoc simulation with on schedule dosing (baseline)
Scenario 1 (delay/re-priming after priming dose of 0.16 mg epcoritamab)
   Cohort 2 (delayed dosing, re-priming not required): Post hoc simulation where intermediate dose is given 8 days after priming dose (not meeting re-priming threshold; 1-day delay from the scheduled intermediate dose), followed by scheduled doses
   Cohort 3 (delayed dosing, re-priming required): Post hoc simulation where re-priming occurs 9 days after priming dose (meeting the re-priming threshold; 2-day delay from the scheduled intermediate dose), followed by scheduled doses
Scenario 2 (delay/re-priming after intermediate dose of 0.8 mg epcoritamab)
   Cohort 2 (delayed dosing, re-priming not required): Post hoc simulation where first full dose is given 14 days after intermediate dose (not meeting re-priming threshold; 7-day delay from the scheduled first full dose), followed by scheduled doses
   Cohort 3 (delayed dosing, re-priming required): Post hoc simulation where re-priming occurs 21 days after intermediate dose (meeting the re-priming threshold; 14-day delay from the scheduled first full dose), followed by scheduled doses
Scenario 3 (Delay/re-priming after a QW full dose of 48 mg epcoritamab)
   Cohort 2 (delayed dosing, re-priming not required): Post hoc simulation where second full dose is given 6 weeks after the first full dose (not meeting re-priming threshold for >6 weeks between full doses; 5-week delay from the scheduled QW dose [second full dose]), followed by scheduled doses
   Cohort 3 (delayed dosing, re-priming required): Post hoc simulation where repriming occurs 7 weeks after the first full dose (meeting the re-priming threshold of >6 weeks between full doses; 6-week delay from the scheduled QW dose [second full dose]), followed by scheduled doses
Scenario 4 (Delay/repriming after a Q2W full dose of 48 mg epcoritamab):
   Cohort 2 (delayed dosing, re-priming not required): Post hoc simulation where full dose is given 6 weeks after full dose on C9D1 (not meeting re-priming threshold for >6 weeks between full doses; 4-week delay from the scheduled Q2W dose), followed by scheduled doses
   Cohort 3 (delayed dosing, re-priming required): Post hoc simulation where repriming occurs 8 weeks after full dose on C9D1 (meeting the re-priming threshold of >6 weeks between full doses, 6-week delay from the scheduled Q2W dose), followed by scheduled doses
Scenario 5 (Delay/re-priming after a Q4W full dose of 48 mg epcoritamab):
   Cohort 2 (delayed dosing, re-priming not required): Posthoc simulation where full dose is given 6 weeks after full dose on C12D1 (not meeting re-priming threshold for >6 weeks between full doses, 2-week delay from the scheduled Q4W dose), followed by scheduled doses
   Cohort 3 (delayed dosing, re-priming required): Posthoc simulation where repriming occurs 10 weeks after full dose on C9D1 (meeting the re-priming threshold of >6 weeks between full doses, 6-week delay from the scheduled Q4W dose), followed by scheduled doses

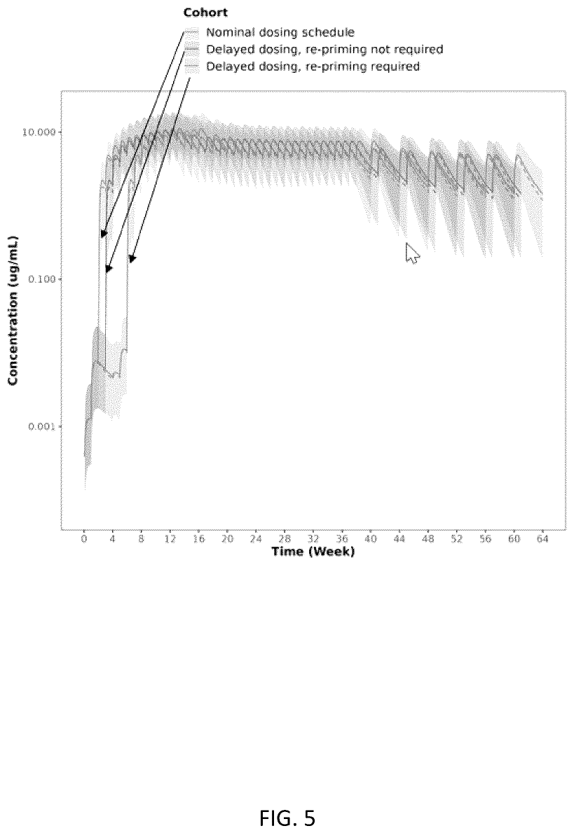

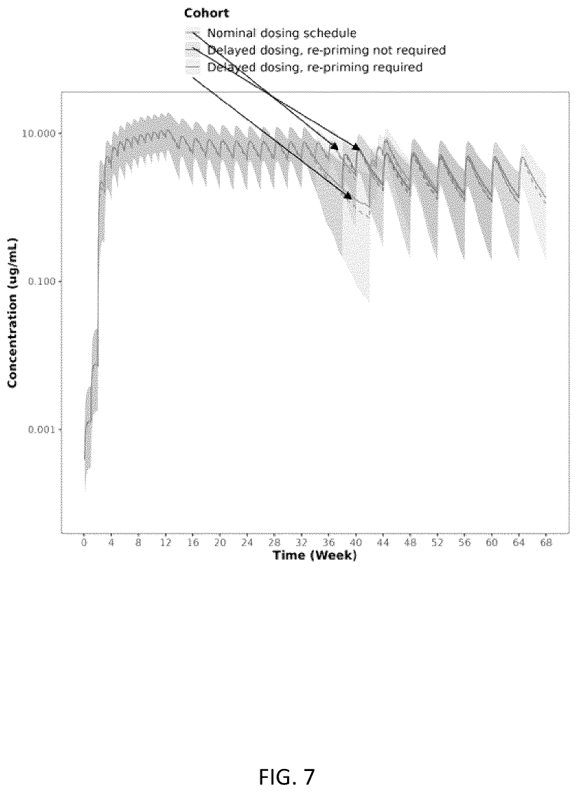

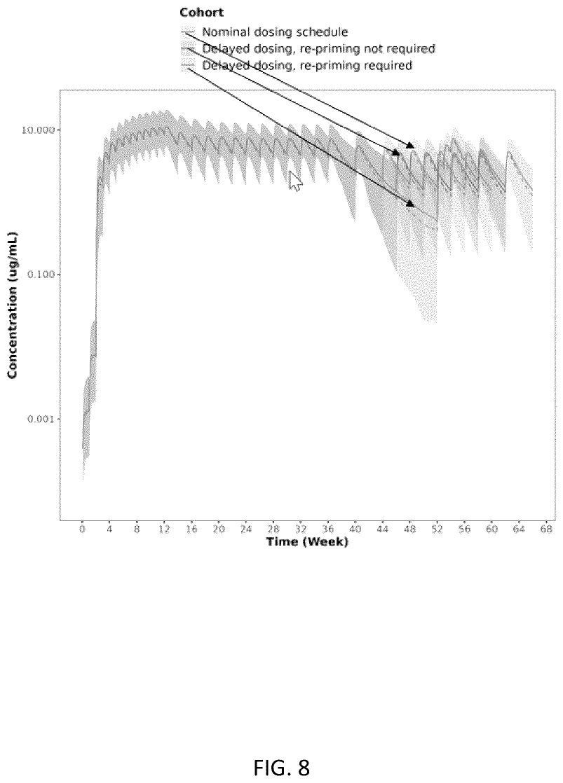

The concentrations were generally maintained for a delayed dose within the proposed time windows. Although the concentration dropped if the second full dose was given 6 weeks after the first full dose (Scenario 2/Cohort 2 in FIG. 5), the concentrations were still maintained well above those after the initial priming and intermediate doses, whereas no substantial decrease in concentrations was observed if a full dose was given within 6 weeks during the Q2W and Q4W dosing schedules (Scenario 4/Cohort 2 in FIG. 7, Scenario 5/Cohort 2 in FIG. 8). Based on the PK simulations, the re-priming windows were adequate.

Repeated Time-to-Event (rTTE) Modeling Approach for CRS

A PK-CRS model was developed to describe the CRS risk. The longitudinal exposure-CRS relationship based on data across a wide range and combinations of priming/intermediate doses from the GCT3013-01 (NCT03625037) and GCT3013-04 (NCT04542824) clinical trials were described using the rTTE model. The recorded times of Grade 2 or higher CRS events were used as event times and were right censored at the end of observation. The hazard function for repeated CRS events was modeled as an effect of epcoritamab plasma concentration. To describe the development of tolerance for CRS hazard following repeated dosing, an inhibitory effect on the hazard was included in a turnover model where the rate of inhibition was stimulated by epcoritamab plasma concentration. Random effects were included on the inhibition component.

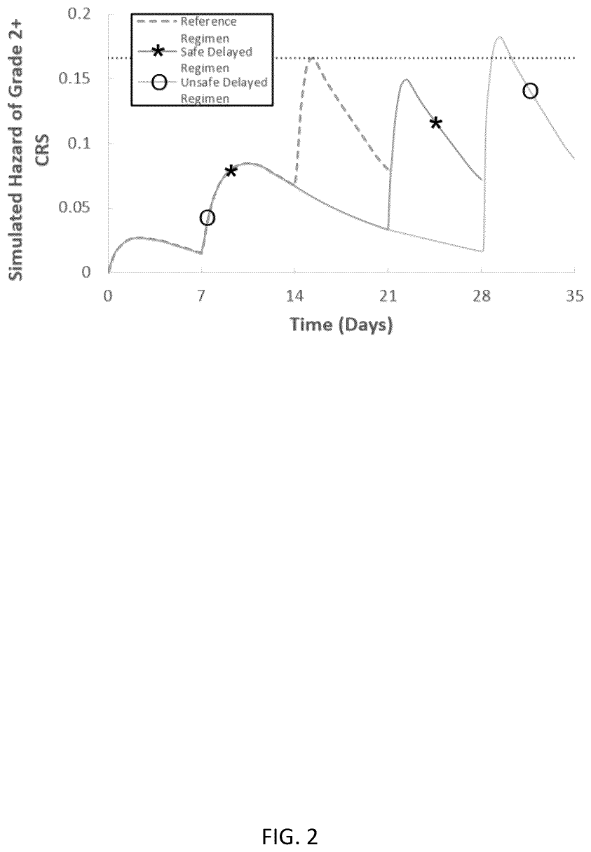

Based on the rTTE PK-CRS model, the instantaneous hazard-time profile for each subject was simulated. The tolerance effect decreases with increasing duration of delay from the last dose administration. Therefore, a longer delay in dosing will lead to a higher hazard of CRS, depending on individual subject's tolerance function and hazard function kinetics. The safe re-priming window is defined as the following (see FIG. 2):
During the step-up dosing phase (ie, intermediate dose, and the first full dose), it was assumed that re-priming was required when the peak instantaneous CRS hazard from the delayed intermediate dose or the first full dose was greater than peaks from the corresponding, scheduled priming/intermediate regimen, or the priming/intermediate/first full dose regimen (reference schedule), respectively. Therefore, the delayed duration was considered safe as long as the peak hazard from the delayed intermediate dose or delayed first full dose was lower than or equal to the peak hazard from reference, scheduled priming/intermediate regimen, or the priming/intermediate/first full dose regimen, respectively.
Due to the low CRS risk following the second full dose onwards, it was assumed that re-priming was required when the peak instantaneous CRS hazard from the delayed full doses was greater than the maximum CRS hazard from both the initial priming dose and the scheduled dose (second full dose or specific full dose after Cycle 1), ie, the reference CRS hazard was the higher peak hazard value from either initial priming dose or the scheduled dose (second full dose or specific full dose after Cycle 1). The delayed duration was considered safe as long as the peak hazard from the delayed full dose was lower than or equal to the reference hazard.

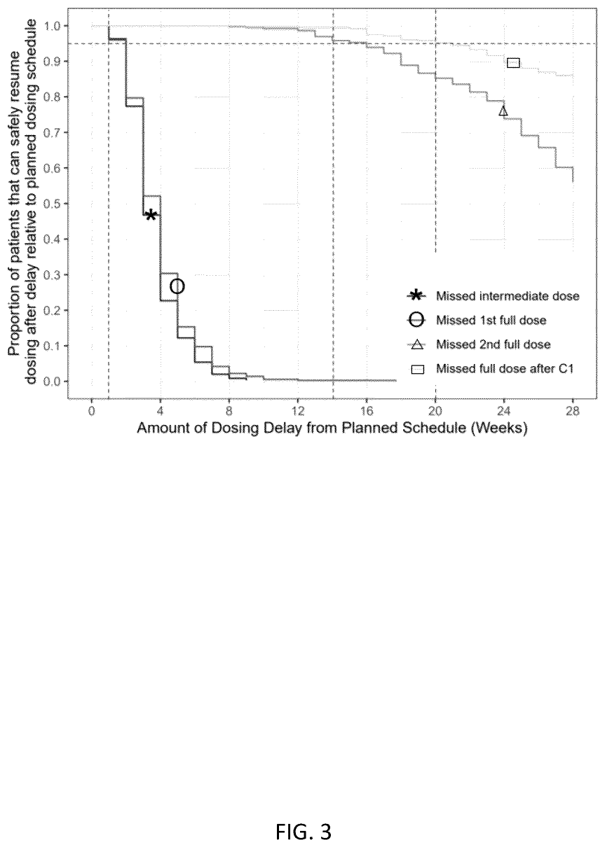

Scenarios of delayed intermediate dose, delayed first full dose, delayed second full dose, delayed full dose after cycle 1 were simulated. For each of these scenarios, a delay of 1 to 28 weeks was simulated. The proportion of subjects who could safely to resume dosing (per definition above) at each specified delay for each scenario is presented in FIG. 3.

Based on the results from simulation using the CRS rTTE model, after missing a scheduled intermediate dose, a delay of ≤1 week was considered safe (ie, >95% of simulated subjects could safely resume the planned intermediate dose without an increase in CRS risk). Similarly, after missing the scheduled first full dose, a delay of ≤1 week could be considered safe. After missing a scheduled second full dose and full doses after Cycle 1, the safe re-priming window was projected to be 14 and 20 weeks, respectively.

### Example 5: Identifying Dose Delays in EPCORE NHL-1

Dose delays were identified in EPCORE NHL-1 study (GCT3013-01; NCT03625037) that were greater than the proposed cut-off durations above for dose delays that requires re-priming. The data is presented in Table 10.

TABLE 10
Events following dose delays in patients in EPCORE NHL-1 (without repriming)

| Duration | Delayed dose | No CRS | Grade 1 CRS | Grade 2 CRS |
|---|---|---|---|---|
| CRS following | SUD 2 delay | ≤8 days between SUD 1 and SUD 2 | | |
| dose delay ≤ | (n = 0) | | | |
| proposed repriming | | N/A | N/A | N/A |
| cut-off | First full dose | ≤14 days between SUD 2 and first full dose | | |
| | delay | | | |
| | (n = 8) | | | |
| | | 50% | 25% | 25% |
| | Other full dose | ≤42 days between full doses | | |
| | delay | | | |
| | QW dosing | 93.5% | 2.7% | 3.7% |
| | (n = 108) | | | |
| | Q2W dosing | 100% | 0 | 0 |
| | (n = 43) | | | |
| | Q4W dosing | 100% | 0 | 0 |
| | (n = 8) | | | |
| CRS following | SUD 2 delay | >8 days between SUD 1 and SUD 2 | | |
| dose delay > | (n = 13) | | | |
| proposed repriming | | 92.3% | 0 | 7.7% |
| cut-off | First full dose | >14 days between SUD 2 and first full dose | | |
| | delay | | | |
| | (n = 2) | | | |
| | | 0 | 50% | 50% |
| | Other full dose | >42 days between full doses | | |
| | delay | | | |
| | QW, Q2W or Q4W | N/A | N/A | N/A |
| | (n = 0) | | | |

N/A = Not applicable

Evaluation of CRS events following dose delays without repriming indicated no increase in the risk of CRS following dose delays that were less than the proposed cutoffs for repriming when compared to overall CRS risk found in EPCORE NHL-1.

SEQUENCE LISTING

| SEQ ID | Description | Sequence |
|---|---|---|
| 1 | CD3 Binding Arm | EVKLVESGGGLVQPGGSLRLSCAASGFTFNTYAMNWVR |
| | 3005a Heavy Chain | QAPGKGLEWVARIRSKYNNYATYYADSVKDRFTISRDD |
| | | SKSSLYLQMNNLKTEDTAMYYCVRHGNFGNSYVSWFA |
| | | YWGQGTLVTVSSASTKGPSVFPLAPSSKSTSGGTAALGC |
| | | LVKDYFPEPVTVSWNSGALTSGVHTFPAVLQSSGLYSLS |
| | | SVVTVPSSSLGTQTYICNVNHKPSNTKVDKRVEPKSCDK |
| | | THTCPPCPAPEFEGGPSVFLFPPKPKDTLMISRTPEVTCV |
| | | VVAVSHEDPEVKFNWYVDGVEVHNAKTKPREEQYNST |
| | | YRVVSVLTVLHQDWLNGKEYKCKVSNKALPAPIEKTIS |
| | | KAKGQPREPQVYTLPPSREEMTKNQVSLTCLVKGFYPSD |
| | | IAVEWESNGQPENNYKTTPPVLDSDGSFLLYSKLTVDKS |
| | | RWQQGNVFSCSVMHEALHNHYTQKSLSLSPG |
| 2 | CD3 Binding Arm | QAVVTQEPSFSVSPGGTVTLTCRSSTGAVTTSNYANWV |
| | 3005a Light Chain | QQTPGQAFRGLIGGTNKRAPGVPARFSGSLIGDKAALTIT |
| | | GAQADDESIYFCALWYSNLWVFGGGTKLTVLGQPKAAP |
| | | SVTLFPPSSEELQANKATLVCLISDFYPGAVTVAWKADS |
| | | SPVKAGVETTTPSKQSNNKYAASSYLSLTPEQWKSHRSY |
| | | SCQVTHEGSTVEKTVAPTECS |
| 3 | CD20 Binding Arm | EVQLVESGGGLVQPDRSLRLSCAASGFTFHDYAMHWVR |
| | 3001d Heavy Chain | QAPGKGLEWVSTISWNSGTIGYADSVKGRFTISRDNAKN |
| | | SLYLQMNSLRAEDTALYYCAKDIQYGNYYYGMDVWG |
| | | QGTTVTVSSASTKGPSVFPLAPSSKSTSGGTAALGCLVK |
| | | DYFPEPVTVSWNSGALTSGVHTFPAVLQSSGLYSLSSVV |
| | | TVPSSSLGTQTYICNVNHKPSNTKVDKRVEPKSCDKTHT |
| | | CPPCPAPEFEGGPSVFLFPPKPKDTLMISRTPEVTCVVVA |
| | | VSHEDPEVKFNWYVDGVEVHNAKTKPREEQYNSTYRV |
| | | VSVLTVLHQDWLNGKEYKCKVSNKALPAPIEKTISKAK |
| | | GQPREPQVYTLPPSREEMTKNQVSLTCLVKGFYPSDIAV |
| | | EWESNGQPENNYKTTPPVLDSDGSFFLYSRLTVDKSRW |
| | | QQGNVFSCSVMHEALHNHYTQKSLSLSPG |
| 4 | CD20 Binding Arm | EIVLTQSPATLSLSPGERATLSCRASQSVSSYLAWYQQKP |
| | 3001d Light Chain | GQAPRLLIYDASNRATGIPARFSGSGSGTDFTLTISSLEPE |
| | | DFAVYYCQQRSNWPITFGQGTRLEIKRTVAAPSVFIFPPS |
| | | DEQLKSGTASVVCLLNNFYPREAKVQWKVDNALQSGN |
| | | SQESVTEQDSKDSTYSLSSTLTLSKADYEKHKVYACEVT |
| | | HQGLSSPVTKSFNRGEC |
| 5 | CD3 Binding Arm | EVKLVESGGGLVQPGGSLRLSCAASGFTFNTYAMNWVR |
| | VH Region | QAPGKGLEWVARIRSKYNNYATYYADSVKDRFTISRDD |
| | | SKSSLYLQMNNLKTEDTAMYYCVRHGNFGNSYVSWFA |
| | | YWGQGTLVTVSS |
| 6 | CD3 Binding Arm | QAVVTQEPSFSVSPGGTVTLTCRSSTGAVTTSNYANWV |
| | VL Region | QQTPGQAFRGLIGGTNKRAPGVPARFSGSLIGDKAALTIT |
| | | GAQADDESIYFCALWYSNLWVFGGGTKLTVL |
| 7 | CD20 Binding Arm | EVQLVESGGGLVQPDRSLRLSCAASGFTFHDYAMHWVR |
| | VH Region | QAPGKGLEWVSTISWNSGTIGYADSVKGRFTISRDNAKN |
| | | SLYLQMNSLRAEDTALYYCAKDIQYGNYYYGMDVWG |
| | | QGTTVTVSS |
| 8 | CD20 Binding Arm | EIVLTQSPATLSLSPGERATLSCRASQSVSSYLAWYQQKP |
| | VL Region | GQAPRLLIYDASNRATGIPARFSGSGSGTDFTLTISSLEPE |
| | | DFAVYYCQQRSNWPITFGQGTRLEIK |

1. A method for treating a CD20 expressing B-cell cancer in a human patient comprising administering epcoritamab in a 28-day priming cycle, the 28-day priming cycle comprising administering one 0.16 mg dose of epcoritamab on Day 1, administering one 0.8 mg dose of epcoritamab on Day 7 or Day 8 at the latest, administering a first 48 mg dose on Day 15, and administering a second 48 mg dose on Day 22, wherein the priming cycle is followed by administering 48 mg doses weekly or every second week, and wherein when
a) the 0.8 mg dose is not administered on Day 8 at the latest, then another dose of 0.16 mg is administered, followed within 7 days by a 0.8 mg dose, prior to administration of the first and second 48 mg doses; and/or
b) the first 48 mg dose is not administered within 14 days from the administration of the 0.8 mg dose, then another dose of 0.16 mg is administered, followed within 7 days by another 0.8 mg dose prior to administration of the first and second 48 mg doses, and/or
c) the second 48 mg dose of the 28-day priming cycle or any of the subsequent 48 mg doses scheduled to be administered weekly or every second week is administered later than 6 weeks from the prior 48 mg dose, then the priming cycle is repeated before administering the 48 mg doses weekly or every second week.

2. The method of claim 1, wherein when the 0.8 mg dose is not administered on day 7 or 8 at the latest during the priming cycle, then a 0.8 mg dose of epcoritamab is administered 7 days after administration of another dose of 0.16 mg, the first 48 mg dose is administered 14 days after administration of the another dose of 0.16 mg, and the second 48 mg dose is administered 21 days after administration of the another dose of 0.16 mg.

3. The method of claim 1, wherein when the first 48 mg dose is administered within 14 days after the administration of the 0.8 mg dose during the priming cycle, the second 48 mg dose is administered 7 days later than the first 48 mg dose.

4. The method of claim 1, wherein when any the 48 mg doses to be administered weekly or every second week is not administered as scheduled, but is administered within 6 weeks from the prior 48 mg dose, then the following 48 mg doses are administered weekly or every second week as scheduled.

5. The method of claim 1, wherein the epcoritamab is administered subcutaneously.

6. The method of claim 1, wherein the 48 mg doses administered weekly or every second week after the priming cycle are administered in a dosing regimen comprising
a) 2 cycles of 28 days, wherein epcoritamab is administered on Days 1, 8, 15 and 22; followed by
b) 6 cycles of 28 days, wherein epcoritamab is administered on days 1 and 15 of cycles 4-9; and
c) subcutaneously administering a dose of 48 mg on Day 1 for all subsequent 28 day cycles.

7. A method for treating a CD20 expressing B-cell cancer in a human patient wherein the patient is scheduled to receive epcoritamab in 28 day cycles and wherein the patient receives a 0.16 mg dose of epcoritamab on Cycle 1, Day 1 and the next scheduled dose is delayed by more than 8 days, dosing is resumed by:
a. subcutaneously administering to the patient another 0.16 mg dose of epcoritamab,
b. subcutaneously administering to the patient a 0.8 mg dose of epcoritamab the following week and,
c. subcutaneously administering a 48 mg dose of epcoritamab for two more weeks before starting Day 1 of the subsequent cycle.

8. A method for treating a CD20 expressing B-cell cancer in a human patient wherein the patient is scheduled to receive epcoritamab in 28 day cycles and wherein the patient receives a 0.8 mg dose of epcoritamab on Cycle 1, Day 8 and the next scheduled dose of epcoritamab is administered 14 days later or less, dosing is resumed by subcutaneously administering a 48 mg dose of epcoritamab and then subsequently continuing with the scheduled dosing, wherein the recommended 28-day dosing schedule is as follows:
a) Cycles 2 and 3, subcutaneously administering a dose of 48 mg on Days 1, 8, 15 and 22;
b) Cycles 4 to 9, subcutaneously administering a dose of 48 mg on Days 1 and 15 of cycles 4-9; and
c) subcutaneously administering a dose of 48 mg on Day 1 for all subsequent cycles.

9. A method for treating a CD20 expressing B-cell cancer in a human patient wherein the patient is scheduled to receive epcoritamab in 28 day cycles and wherein the patient receives a 0.8 mg dose of epcoritamab on Cycle 1, Day 8 and the next scheduled dose of epcoritamab is administered more than 14 days after the 0.8 mg dose, the dosing is resumed by:
a. subcutaneously administering to the patient a dose of 0.16 mg of epcoritamab,
b. subcutaneously administering to the patient a dose of 0.8 mg of epcoritamab the following week, and,
c. subcutaneously administering two weekly doses of 48 mg of epcoritamab before starting Day 1 of the subsequent cycle.

10. A method for treating a CD20 expressing B-cell cancer in a human patient wherein the patient is scheduled to receive epcoritamab in 28 day cycles and wherein the patient receives a first 48 mg dose of epcoritamab on Cycle 1, Day 15, and the next scheduled dose of 48 mg of epcoritamab is administered 6 weeks or less after the first 48 mg dose, the 28-day dosing schedule is continued as follows:
a) Cycles 2 and 3, subcutaneously administering a dose of 48 mg on Days 1, 8, 15 and 22;
b) Cycles 4 to 9, subcutaneously administering a dose of 48 mg on Days 1 and 15 of cycles 4-9; and
c) subcutaneously administering a dose of 48 mg on Day 1 for all subsequent cycles.

11. (canceled)

12. The method of claim 1, comprising administering oral or intravenous corticosteroids to the patient for 4 consecutive days in connection with each dose of epcoritamab.

13. The method of claim 12, wherein the corticosteroids are administered in connection with each dose of epcoritamab until at least two consecutive doses of 48 mg epcoritamab have been administered.

14. The method of claim 12, wherein the corticosteroids are administered on the same day epcoritamab is administered and on the three following days.

15. The method of claim 12, comprising administering oral or intravenous corticosteroids to the patient 30 to 120 minutes before each dose of epcoritamab.

16. The method of claim 12, wherein the corticosteroids are selected from the group consisting of 100 mg oral or intravenous prednisolone and 15 mg oral or intravenous dexamethasone or an equivalent.

17. The method of claim 1, further comprising administering oral or intravenous diphenhydramine or acetaminophen to the patient 30 to 120 minutes before each of the 4 epcoritamab doses in the priming cycle.

18. A method for treating a CD20 expressing B-cell cancer in a human patient wherein the patient is considered to be at risk for antifungal or antiviral infections, comprising prophylactically administering an antibiotic, antiviral or antifungal therapy prior to starting treatment with epcoritamab.

19. The method of claim 18, wherein the patient is at risk of an antifungal infection caused by Pneumocystis jirovecii, the method comprising orally administering to the patient trimethoprim/sulfamethoxazole 160 mg/800 mg every other day.

20. The method of claim 18, wherein the patient is at risk for recurrent antiviral infection, comprising administering an antiviral therapy such as acyclovir for recurrent herpes virus infections, or a nucleoside/nucleotide analogue such as tenofovir disoproxil fumarate, tenofovir alafenamide, or entecavir for chronic hepatitis B virus infections.

21. (canceled)
